(19) United States
(12) Patent Application Publication
(10) Pub. No.: US 2025/0204762 A1
(43) Pub. Date: Jun. 26, 2025

(54) VIDEO SIGNAL TRANSLATOR FOR USE IN MEDICAL APPLICATIONS

(71) Applicant: Boston Scientific Scimed, Inc., Maple Grove, MN (US)

(72) Inventors: Kirsten VIERING, Newton, MA (US); Louis J. BARBATO, Franklin, MA (US)

(73) Assignee: Boston Scientific Scimed, Inc., Maple Grove, MN (US)

(21) Appl. No.: 18/985,554

(22) Filed: Dec. 18, 2024

Related U.S. Application Data
(60) Provisional application No. 63/613,306, filed on Dec. 21, 2023.

(51) Int. Cl. A61B 1/045; A61B 1/06; H04N 7/01; H04N 25/71; H04N 25/772

(52) U.S. Cl. CPC A61B 1/045; A61B 1/0655; A61B 1/0676; H04N 7/013; H04N 25/71; H04N 25/772

(57) ABSTRACT

Disclosed embodiments relate to a medical system including a video signal translator. The video signal translator is configured to: receive a first video signal from an imaging device, the first video signal having a first video signal format; translate the first video signal from the first video signal format to a second video signal format that is different from the first video signal format; and output, to a control unit, the translated video signal as a second video signal. The control unit is further configured to receive the second video signal and process the second video signal for output to an electronic display.

### CROSS-REFERENCE TO RELATED APPLICATIONS

This application claims the benefit of priority under 35 U.S.C. § 119 from U.S. Provisional Application No. 63/613,306, filed Dec. 21, 2023, which is incorporated by reference herein in its entirety.

### TECHNICAL FIELD

Various aspects of this disclosure relate generally to medical devices, for example scopes such as endoscopes or bronchoscopes, that include imaging elements. More specifically, aspects of this disclosure relate to techniques for translating video signals captured by such medical devices.

### BACKGROUND

Endoscopes have attained great acceptance within the medical community since they provide a means for performing procedures with minimal patient trauma while enabling the physician to view the internal anatomy of the patient. Numerous endoscopes have been developed and categorized according to specific applications, such as cystoscopy, colonoscopy, bronchoscopy, upper GI endoscopy, and others.

An endoscope usually has an elongated tubular shaft, having a video camera or a fiber optic lens assembly at its distal end. The shaft is connected to a handle. Viewing is usually possible via an external display. Various surgical tools may be inserted through a working channel in the endoscope for performing different surgical procedures. Endoscopes, such as colonoscopes, typically have a front camera for viewing the internal organ, such as the colon, an illuminator, a fluid injector for cleaning the camera lens (and sometimes also the illuminator), and a working channel for insertion of surgical tools, for example, for removing polyps found in the colon. Often, endoscopes also have fluid injectors for cleaning a body cavity, such as the colon, into which they are inserted. The illuminators commonly used include fiber optics which transmit light to the endoscope tip section and light-emitting diodes (LEDs) at the endoscope tip section.

Current endoscopes typically implement an imaging system through a camera sensor at a tip section of the endoscope, such as a charge coupled device (CCD) sensor or a complementary metal-oxide semiconductor (CMOS) sensor. The imaging system ultimately connects to a control unit, which can include further capabilities such as outputting images or video on a display or adjusting light sources connected to or within the endoscope.

But imaging systems within some types of endoscopes can be incompatible with other types of control units. Accordingly, there is a need in the art for image processing devices, systems, and methods capable of translating a camera signal to be compatible with different control units.

### SUMMARY

Aspects of the disclosure relate to, among other things, systems, devices, and methods for video translation of an image signal of a medical device. For example, certain aspects implement a video signal translator that can either be housed within the medical device (such as an endoscope) or an adapter, such as a pigtail, or other type of adapter.

In some aspects, the embodiments disclosed herein relate to a medical system including a video signal translator. The video signal translator is configured to: receive a first video signal from an imaging device, the first video signal having a first video signal format; translate the first video signal from the first video signal format to a second video signal format that is different from the first video signal format; and output, to a control unit, the translated video signal as a second video signal. The control unit is configured to receive the second video signal and process the second video signal for output to an electronic display.

In some aspects, the embodiments disclosed herein relate to a medical system that further includes a medical device including a distal tip portion, the distal tip portion including the imaging device configured to output the first video signal in the first video signal format.

In some aspects, the video signal translator is positioned within a handle of the medical device.

In some aspects, the embodiments disclosed herein relate to a medical system in which the first video signal format is an analog video format, the second video signal format is a digital video format, the video signal translator includes an analog-to-digital converter. In some aspects, translating the first video signal includes: applying the analog-to-digital converter to the first video signal, and receiving, from the analog-to-digital converter, the second video signal.

In some aspects, the embodiments disclosed herein relate to a medical system in which the imaging device is a Charge Coupled Device (CCD), and in which the second video signal is compatible with a signal from a metal oxide semiconductor (CMOS).

In some aspects, the embodiments disclosed herein relate to a medical system in which the first video signal includes a first frame rate. The medical system further includes a processor that is configured to adjust the first frame rate of the first video signal to a second frame rate of the second video signal. The second frame rate is different than the first frame rate.

In some aspects, the embodiments disclosed herein relate to a medical system that includes a lighting source configured to cause light to be emitted at the distal tip portion. In some aspects, translation of the first video signal to the second video signal includes: causing the lighting source to emit, at a first time, light having a first primary color; capturing a first frame of video; causing the lighting source to emit, at a second time, light having a second primary color different from the first primary color; capturing a second frame of video; and constructing the second video signal from the first frame of video and the second frame of video.

In some aspects, the embodiments disclosed herein relate to a medical system that further includes a processor that is configured to deconstruct the first video signal into separate primary color components and construct the second video signal with the primary color components into a mosaic. Each pixel value of the second video signal includes a single primary color component.

In some aspects, embodiments disclosed herein relate to a medical system in which the first video signal format is a digital signal format, the second video signal is an analog signal format; the video signal translator includes a digital-to-analog converter, and in which translating the first video signal includes: applying the digital-to-analog converter to the first video signal, and receiving, from the digital-to-analog converter, the second video signal.

In some aspects, the embodiments disclosed herein relate to a medical system in which the imaging device is a CMOS device. The second video signal is compatible with a signal from a CCD.

In some aspects, the embodiments disclosed herein relate to a medical system in which the first video signal format is an analog video signal format with a first signaling type, and the second video signal format is a second analog video signal format having a second signaling type that is different the first signaling type.

In some aspects, the embodiments disclosed herein relate to a medical system in which each of the first video signal format and the second video signal format conform to a Low Voltage Differential Signaling (LVDS) format or a Mobile Industry Processor Interface (MIPI) format.

In some aspects, the embodiments disclosed herein relate to a medical system further including an adaptor having the video signal translator. The adaptor is attachable to the medical device.

In some aspects, the embodiments disclosed herein relate to a medical system that is an endoscope and in which the adaptor includes a light coupling configured to propagate light from a lighting source to the endoscope.

In some aspects, the embodiments disclosed herein relate to a medical system in which the control unit is configured to process the second video signal prior to outputting the second video signal to an electronic display.

In some aspects, the embodiments disclosed herein relate to a medical system that includes a medical device including a shaft having a distal tip portion, the distal tip portion including an imaging device configured to output a first video signal in a first video signal format; and a video signal translator. The video signal translator is configured to: receive the first video signal from the imaging device; translate the first video signal from the first video signal format to a second video signal format that is different from the first video signal format. The medical device includes a control unit configured to: receive the translated video signal from the video signal translator, and process the translated video signal into an image processed video signal for output.

In some aspects, the embodiments disclosed herein relate to a medical system in which the first video signal format is an analog video format, the second video signal format is a digital video format, the video signal translator includes an analog-to-digital converter, and in which translating the first video signal includes applying the analog-to-digital converter to the first video signal, and receiving, from the analog-to-digital converter, the second video signal.

In some aspects, the embodiments disclosed herein relate to a medical system in which the first video signal format is a digital signal format, the second video signal is an analog signal format, the video signal translator includes a digital-to-analog converter, and in which translating the first video signal includes: applying the digital-to-analog converter to the first video signal, and receiving, from the digital-to-analog converter, the second video signal.

In some aspects, the embodiments disclosed herein relate to method that includes receiving, from an imaging device within the medical device, a first video signal in a first video signal format; translating the first video signal from the first video signal format to a second video signal format that is different from the first video signal format; outputting, to a control unit, the translated video signal as a second video signal; receiving, at the control unit, the second video signal; processing the second video signal to an image processed video signal, via the control unit; and displaying, on an electronic display, the image processed video signal.

In some aspects, the embodiments disclosed herein relate to a medical system in which the first video signal format is an analog video format, the second video signal format is a digital video format, and in which translating the first video signal includes applying an analog-to-digital converter to the first video signal, and receiving, from the analog-to-digital converter, the second video signal.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIG. 2 is a diagram of a system for video translation for an endoscope, according to aspects of this disclosure.

FIG. 3 is a diagram of an analog to digital conversion system for an endoscope, according to aspects of this disclosure.

FIG. 4 is a diagram of a digital to analog conversion system for an endoscope, according to aspects of this disclosure.

FIG. 5 is a diagram of charge-coupled device (CCD) to complementary metal oxide semiconductor (CMOS) conversion system for an endoscope, according to aspects of this disclosure.

FIG. 6 is a diagram of CMOS to CCD conversion system for an endoscope, according to aspects of this disclosure.

FIG. 7 is a diagram of example analog signaling schemes for use in an endoscope, according to aspects of this disclosure.

FIG. 8 is a diagram of an example circuit for a Mobile Industry Processor Interface (MIPI) to Low Voltage Differential Signaling (LVDS) conversion system for an endoscope, according to aspects of this disclosure.

FIG. 9 is a diagram of an example circuit for a LVDS to MIPI conversion system for an endoscope, according to aspects of this disclosure.

FIG. 10 is a flow diagram of an example frame rate conversion system for an endoscope, according to aspects of this disclosure.

FIG. 11 is a diagram of inputs and outputs of a video de-mosaicing system for an endoscope, according to aspects of this disclosure.

FIG. 12 is a diagram of an example monochrome to color conversion system for an endoscope, according to aspects of this disclosure.

FIG. 13 is a diagram of exemplary inputs and outputs of a monochrome to color conversion system for an endoscope, according to aspects of this disclosure.

FIG. 14 is a diagram of an example mechanical adaptor system for use with an endoscope and a signal translator, according to aspects of this disclosure.

FIG. 15 is a diagram of an example mechanical adaptor system for use with an endoscope and a signal translator, according to aspects of this disclosure.

FIG. 16 is a diagram of an example mechanical adaptor system for use with an endoscope and a signal translator, according to aspects of this disclosure.

FIG. 17 is a diagram of an example mechanical adaptor system for use with an endoscope and a signal translator, according to aspects of this disclosure.

FIG. 18 depicts an example computing device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Aspects of this disclosure relate to video signal translators that can translate image or video signals associated with medical devices, such as endoscopes. Disclosed techniques facilitate wider compatibility between endoscopes and control systems while maintaining a compact package. For example, a camera used in a first endoscope can appear to a control unit like the camera of a second endoscope with different electronic components.

As discussed further herein, disclosed video signal translators may be positioned anywhere within or outside of an endoscope or the related system. For instance, a signal translator may be embedded within the endoscope itself, or provided in an adaptor that is connected to the endoscope.

Examples of signal translation include, but are not limited to, translation between analog to digital or vice versa, translation of signals compatible with a complementary metal-oxide semiconductor (CMOS) to signals compatible with a charge coupled device (CCD) or vice versa, between different types of analog signals, or between different types digital signals. In some aspects, additional processing can be performed before and/or after translation. Non-limiting examples of such processing include scaling, cropping, and adjusting a color space. Disclosed signal translators can also control light sources as appropriate, for instance to facilitate these signal conversions, such as causing a light source to illuminate at a particular time and duration to enable conversion from monochrome to color.

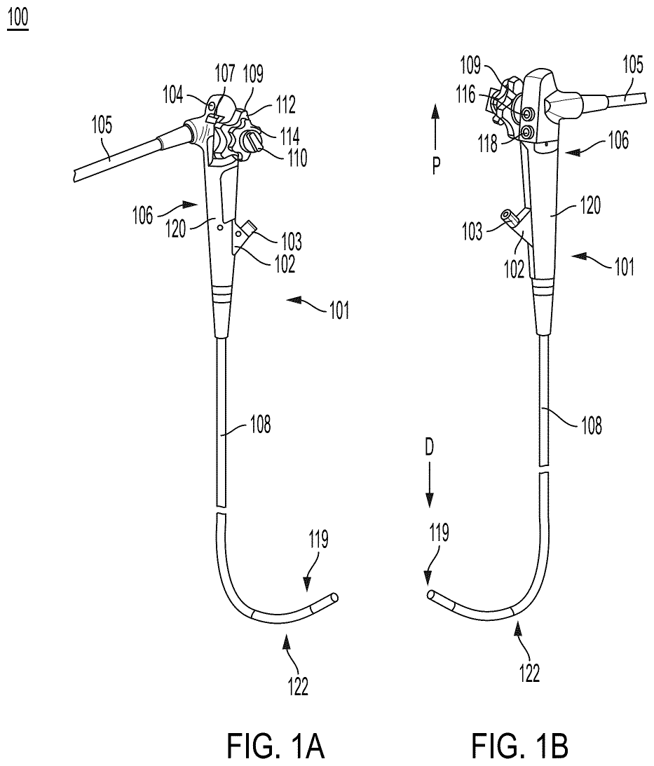

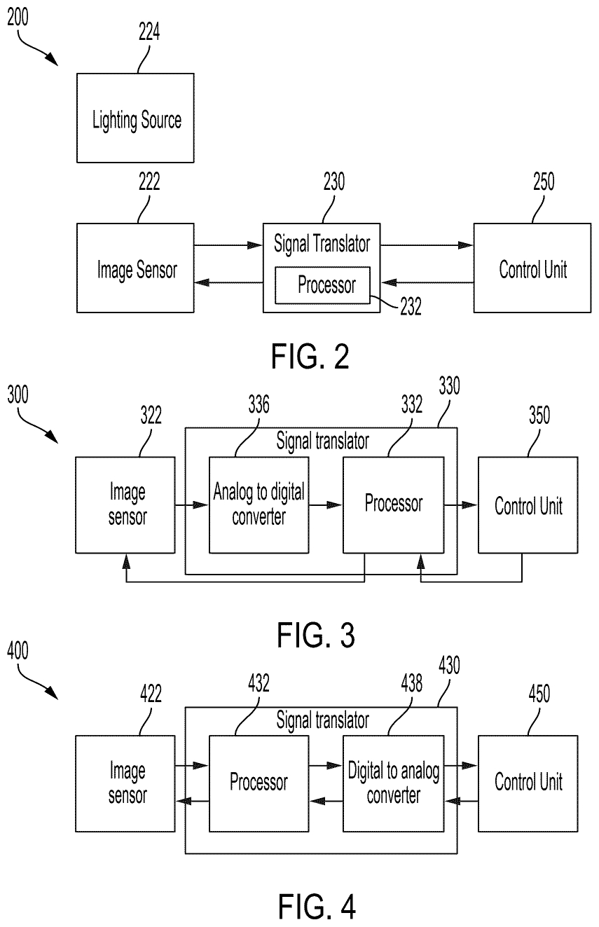

Turning now to the figures, FIGS. 1A and 1B show perspective views of an exemplary endoscope system 100. Endoscope system 100 may include an endoscope 101 and other system components (not shown), such as a controller, a light source, a source of suction and/or irrigation, etc. Endoscope 101 may include a handle assembly 106 and a flexible tubular shaft 108. The handle assembly 106 may include a biopsy port 102, a biopsy cap 103, an image capture button 104, an elevator actuator 107, a first locking lever 109, a second locking lever 110, a first control knob 112, a second control knob 114, a suction button 116, an air/water button 16, a handle body 120, and an umbilicus 105. All of the actuators, elevators, knobs, buttons, levers, ports, or caps of endoscope 101, such as those enumerated above, may serve any purpose and are not limited by any particular use that may be implied by the respective naming of each component used herein. The umbilicus 105, which typically is part of the endoscope 101, may extend from handle body 120 to one or more auxiliary devices, such as a control unit 250 (shown schematically in FIG. 2, for example), water/fluid supply, and/or vacuum source. Umbilicus 105 therefore may transmit signals between endoscope 101 and the control unit 250, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 16 control valves for suction and fluid supply (e.g., air and water), respectively. Shaft 108 may terminate at distal tip 119. Shaft 108 may include an articulation section 122 for deflecting distal tip 119 in up, down, left, and/or right directions. Knobs 112 and 114 may be used for controlling such deflection, and locking levers 109 and 110 may lock knobs 112 and 114, respectively, in desired positions. Handle body 120 may be tapered and may narrow as the handle extends distally such that the profile of handle body 120 is smaller at its distal end than at its proximal end.

Although the term endoscope may be used herein, it will be appreciated that other devices, including, but not limited to, other types of scopes, endoscopes, and medical devices, such as cholangioscopes, duodenoscopes, colonoscopes, ureteroscopes, bronchoscopes, laparoscopes, sheaths, catheters, or any other suitable delivery device or medical device may be used in connection with the devices of this disclosure, and the devices, systems, and methods discussed below may be incorporated into any of these or other medical devices.

FIG. 2 is a diagram of a system 200 for video translation for an endoscope, according to aspects of this disclosure. In the example depicted in system 200, a first video signal is received from image sensor 222. In some cases, lighting source 224 causes a subject to be illuminated by lighting source 224 during the capturing of the first video signal. The first video signal is transmitted in a first video format, e.g., optically or electrically, to signal translator 230. In turn, signal translator 230 converts the signal from the first video format to a second video format. The translated signal is then output to a control unit 250, where it can be visualized or displayed on a display or transmitted to another device.

The components of system 200 can be positioned anywhere in endoscope system 100 or in an adaptor for use with endoscope system 100. For instance, system 200 can be embedded in endoscope system 100 including, for example, endoscope 101, within handle assembly 106, within distal portion 119 of flexible tubular shaft 108, umbilicus 105, or within tip portion 119, and/or any other portion of endoscope system 100. In some cases, components of system 200 can be separated and various components placed in a different positions within or outside of endoscope system 100 and interconnected, for example, electrically or optically.

Examples of image sensor 222 include, but are not limited to an image sensor, camera, optical fiber, and a lens assembly with image sensor. Examples of lighting source 224 include, but are not limited to a Light Emitting Diode (LED) or an optical fiber. In some examples, image sensor 222 is located at a tip portion of an endoscope. In this case, image sensor 222, especially in small diameter endoscopes, may be only an image sensor, such as a CCD or CMOS sensor, without any other image processing components and may output an image signal which is processed by signal translator 230.

For example, referring back to FIGS. 1 and 2, one or more of imaging device 222 and lighting source 224 may be located within distal tip 119. In some examples, distal tip 119 may include a front-facing imaging device 222, may include a front-facing imaging device 222 and a side-facing imaging device 222, may include only a side-facing imaging device 222, may include a front-facing imaging device 222 and two side-facing imaging devices 222, or any other combination of imaging devices at distal tip 119. A side-facing imaging device 222 and the lighting source 224 may face radially outward, perpendicularly, approximately perpendicularly, or otherwise transverse to a longitudinal axis of shaft 108 and distal tip 119. A front-facing or forward-facing imaging device 222 or lighting source 224 may face approximately along or parallel to a longitudinal axis of distal tip 119 and shaft 108. In some examples, imaging device 222 may be a charge coupled device (CCD) image sensor, a complementary metal-oxide semiconductor (CMOS) image sensor, and/or any other element for converting light into electrons to be sent as an electrical signal.

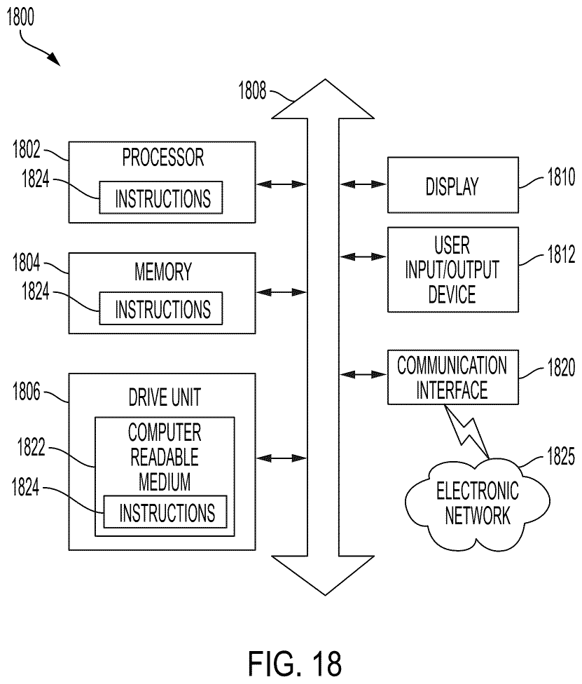

Signal translator 230 can connect to control unit 250 and image sensor 222. Signal translator 230 can include one or more processors 232. Processor 232 can be a general purpose processor, microcontroller, signal processor, application specific integrated circuit (ASIC), Field Programmable Gate Array (FPGA), or other device. An example processor is processor 1802 depicted in FIG. 18.

Processor 232 can be configured to perform any functionality described herein, such as interpolation, decimation, color space transformation, filtering, and/or other adjustments. Processor 232 can perform image processing that may be needed to make the camera used in the endoscope compatible with a particular control unit. For instance, processor 232 can adjust pixel counts by appropriate image processing and scaling. Scaling can include increasing or decreasing resolution, zooming in, or zooming out.

Control unit 250 may be capable of interfacing with endoscope 101, directly or via signal translator 230, to provide power and/or instructions for imaging device(s) 222 and/or light source 224. Control unit 250 may also control one or more other aspects of endoscope 101, such as, for example, the application of suction, the deployment or delivery of fluid, and/or the movement of distal tip 119. Control unit 250 may be powered by an external source such as an electrical outlet. In addition, the control unit 250 may include or otherwise be coupled to one or more buttons, knobs, touchscreens, or other user interfaces to control the imaging device 222, light source 224, and other features of endoscope 101. The control unit 250 may be housed in the handle 106 itself or in a separate apparatus.

Control unit 250 may be configured to set or control one or more illumination and imaging parameters. For example, control unit 250 may set or control an illumination level for each light source 224, gain level for each imaging device 222, exposure time for each imaging device 222, frame rate of each imaging device 222, maximum or target values for any of the illumination and imaging parameters, and/or any other parameter associated with imaging device 222 and/or light source 224.

In some examples, control unit 250 may be configured to execute one or more algorithms using one or more illumination and imaging parameters, for example to automatically adjust an illumination level of one or more of light sources 224 and/or automatically adjust one or more parameters of imaging device(s) 222. For example, control unit 250 may set or select an illumination level for one or more light sources 224 based on data received from one or more imaging devices 222.

Control unit 250 may include electronic circuitry configured to receive, process, and/or transmit data and signals between endoscope 101 and one or more other devices. For example, control unit 250 may be in electronic communication with a display configured to display images based on image data and/or signals processed by control unit 250, which may have been generated by imaging device(s) 222 of endoscope 101. Control unit 250 may be in electronic communication with the display in any suitable manner, either via wires or wirelessly. The display may be manufactured in any suitable manner and may include touch screen inputs and/or be connected to various input and output devices such as, for example, mouse, electronic stylus, printers, servers, and/or other electronic portable devices. Control unit 250 may include software and/or hardware that facilitates operations such as those discussed above. For example, control unit 250 may include one or more algorithms, models, or the like for executing any of the methods and/or systems discussed in this disclosure. Control unit 250 may be configured to automatically adjust the illumination value applied to one or more light source(s) 224, and automatically adjust the gain and the frame rate applied to the one or more imaging device(s) 222.

In operating endoscope system 100, a user may use their left hand to hold handle assembly 106 while the right hand is used to hold accessory devices and/or operate one or more of the actuators of handle assembly 106, such as first and second control knobs 112, 114 and first and second locking levers 109, 110. The user may grasp the handle assembly 106 by wrapping the user's hand around handle body 120. When grasping handle body 120, the user may use the left thumb to operate first and second control knobs 112, 114 and the elevator actuator 107 (through rotation about their respective axes), and may use a left-hand finger to operate the image capture button 104, the suction button 116, and/or the air/water button 118 (each by pressing). The user may rotate the handle assembly 106 (e.g., by moving his/her wrist) in order to rotate shaft 108 about a longitudinal axis of shaft 108 and position distal tip 119 at a target area within a patient's body.

The user may actuate button 104 to initiate video display from the imaging device, take an image (such as a digital image) with the imaging device, and/or take any other action associated with the imaging device. During operation, the user may visualize the video feed from the imaging device on an electronic display. The real-time signal from the imaging device may be processed by control unit 250 and output to the electronic display. The electronic display may be one or more electronic displays such as, for example, a monitor, television, tablet, smartphone, portion of control unit 250, virtual reality display, or other display device. For example, in operation of endoscope 101, a user may first actuate imaging button 104 to either initiate capturing of an image or the start of a video recording using imaging device 222. In some examples, the user may also actuate lighting source 224 to illuminate the field of view of imaging device 222. The imaging device 222 may then receive photons of light within its field of view at an image sensor of imaging device 222, and may transmit a raw analog signal and/or a digital signal.

FIG. 3 is a diagram of an analog to digital conversion system 300 for an endoscope, according to aspects of this disclosure. System 300 includes image sensor 322, signal translator 330, and control unit 350. Image sensor 322 can implement substantially similar functionality as image sensor 222. Signal translator 330 includes analog to digital (A/D) converter 336, which is configurable to convert analog signals received from image sensor 322 to digital signals for processing by control unit 350. Signal translator 330 can also format digital data into a number and type of lanes that is expected by control unit 350.

While not depicted, system 300 can include a lighting source, e.g., lighting source 224. Non-limiting examples of the functionality that can be implemented by signal translator 330 include analog to digital (A/D) conversion, video or image processing, data formatting such as serialization or parallelization, and so forth.

In an example, signal translator 330 receives a first video signal from imaging sensor 322 in analog format. Signal translator 330 translates the first video signal from the first video signal format to a second video signal format that is a digital video signal format by using analog to digital (A/D) converter 336. Specifically, signal translator 330 applies the analog to digital (A/D) converter 336 to the first video signal. Analog to digital (A/D) converter 336 then outputs the translated signal as a second video signal in the second format.

In an aspect, A/D converter 336 can include a front end that is designed to minimize artifacts from anti-aliasing. In this case, a comparator within A/D converter 336 can be used to determine a trigger point from the incoming analog signal. For example, a reference level of the comparator can be adjusted such that the trigger point occurs slightly before an analog trigger point. In so doing, the processor (or FPGA) is ready to expect analog to digital data. When a trigger point is detected, the processor (FPGA) can determine timing parameters within the analog signal to begin the conversion.

Additionally or alternatively, control signals can be passed from the control unit 350 to and from the signal translator such as synchronization signals or user inputs, for example, for adjusting a gain or exposure time of an image sensor or controlling a brightness of a lighting source. Additionally or alternatively, A/D converter 336 may include circuitry to detect and generate line and frame sync information.

The signal translator 330 can be constructed with various electronic components, can be partially or wholly implemented by a Field Programmable Gate Array (FPGA), or an application specific integrated circuit (ASIC). If an FPGA or ASIC, algorithms can be added to communicate with the image sensor that may not be the original intended sensor for a particular control unit 350. The FPGA or ASIC can then also serve as a translator for image sensor control such as gain, region of interest, pixel binning, exposure, and other operations.

In some aspects, the digitized analog data is sent to the control unit 350 without significant, or any, digital processing. If the output of A/D converter 336 matches the expected input of the control unit 350, then the signal translator 330 can serve as a pass-through repeater. In this case, various components of signal translator 330 may not be needed.

In some aspects, signal translator 330 can include one or more processors 332. Processor 332 is configurable to perform operations as described with respect to processor 232 depicted in FIG. 2. Processor 332 can transmit a fully formed image to control unit 350.

In some aspects, processor 332 can act as a data formatter and/or translator. For instance, processor 332 can reformat or process the digitized data into a format expected by the control unit 350. For example, if the control unit 350 expects a serialized digital data stream, and the A/D converter 336 outputs the data in a parallel stream, then the signal translator 330 can serialize the data from A/D converter 336 and transmit the serialized data to the control unit 350. The processor 332 can send one or more control signals from the control unit 350 to control image sensor 322. Examples of controls signals include, but are not limited to, signals to cause image sensor 322 to capture an image, to stop capturing images, and so forth. Accordingly, signal flow between processor 330 and image sensor 322 can be bidirectional.

In some aspects, processor 332 is used to adjust the raw pixel data. For example, it may boost red colored pixels by a defined gain. Processor 332 can be used to up-scale or down-scale the image to the image size expected by the control unit 350. For example, output from a 400×400 pixel image sensor can be scaled down to 250×250 pixels. Examples of algorithms that can be employed by processor 332 include, but are not limited to, bilinear, bicubic, and Lanczos. In a simplified example, processor 332 can be programmed to strategically drop or discard various pixels of the image to facilitate downscaling.

In some aspects, processor 332 can crop pixel data. For instance, processor 332 can crop an image, or a frame of video to a desired field of view. For example, a 120 degree field of view image sensor may be cropped to a 90 degree field of view.

In some aspects, processor 332 can generate timing signals and/or blanking signals expected by control unit 350. More generally, processor 332 simulates any camera signals that are expected by the control unit 350 to maintain interoperability between different combinations of endoscopes and control units. For instance, the digital signal output from processor 332 can be Low Voltage Differential Signaling (LVDS), Mobile Industry Processor Interface (MIPI), Pulse Width Modulation (PWM), or any other signal.

FIG. 4 is a diagram of a digital to analog conversion system 400 for an endoscope, according to aspects of this disclosure. System 400 includes image sensor 422, signal translator 430, and control unit 450. Image sensor 422 can implement substantially similar functionality as image sensor 222. While not depicted, system 400 can include a lighting source, e.g., lighting source 224.

Signal translator 420 includes digital to analog (D/A) converter 438. D/A converter 438 receives a digital signal from image sensor 422 and converts the digital signal into analog format. In an example, signal translator 430 receives, from image sensor 422, a first video signal in digital format. The signal translator 430 applies the D/A converter 438 to the first video signal. The D/A converter 438 outputs the translated signal as an analog signal.

Upon conversion, D/A converter 438 can consider timing needs of the control unit 450. As such, in some cases, signal translator 430 includes an FPGA or ASIC to create the required sync signal and blanking signal for the control unit 450. Similarly, signal translator 430 is configurable to ensure that other requirements such as rise and fall times, or transition times, are met.

In some aspects, signal translator 430 can perform processing of the digitized signal received from image sensor 422 prior to conversion to analog format. For instance, as depicted, signal translator 430 can include one or more processors 432. Processor 432 is configurable to perform operations as described with respect to processor 232 depicted in FIG. 2.

For instance, in some cases, signal translator 430 can reorganize the output sequence of pixels. For instance, if the signal expected by control unit 450 does not meet a pixel order received from image sensor 422, then adjustments are made. For instance, image sensor 422 may output a sequence of pixels: RGRG . . . , GBGBGBG (where R indicates red, G indicates green, and B indicates blue in a RGB color space). But the control unit 450 may expect a sequence with blocks of identical color from sequential pixels such as RRR . . . , GGG . . . , and BBB. . . . In this case, a memory is used to store multiple pixels or frames of data.

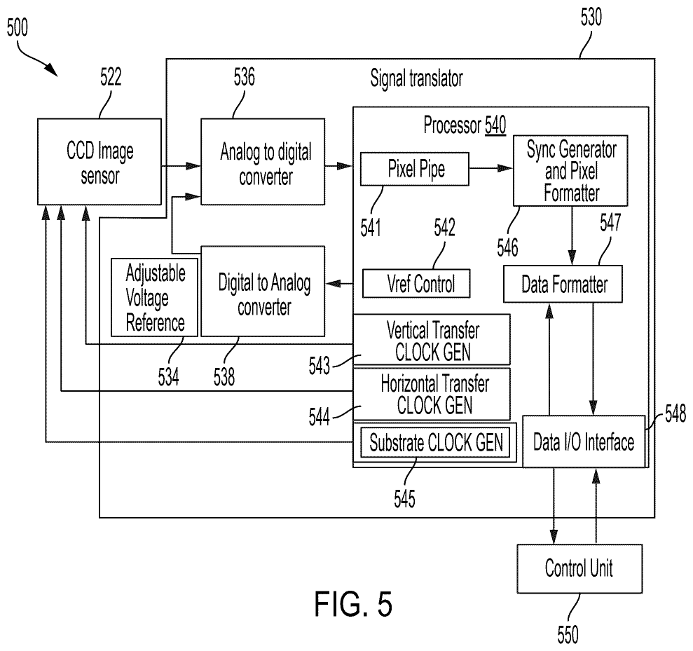

FIG. 5 is a diagram of charge-coupled device (CCD) to complementary metal oxide semiconductor (CMOS) conversion system 500 for an endoscope, according to aspects of this disclosure. In the example depicted, signal translator 530 receives a CCD image signal from CCD image sensor 522 and converts the signal to be compatible with an expected input of a CMOS signal to control unit 550.

Signal translator 530 includes D/A converter 538, A/D converter 536, adjustable voltage reference 534, and processor 540. For discussion purposes, CCD image sensor 522 is assumed to output an image signal in analog form. However, system 500 can operate with a CCD image sensor that outputs a digital signal. In this case, an analog to digital converter is not needed and would be bypassed.

Processor 540 includes pixel pipe 541, Vref control 542, vertical transfer clock generator 543, horizontal transfer clock generator 544, substrate clock generator 545, sync generator and pixel formatter 546, data formatter 547, and data input/output (I/O) interface 548. Processor 540 can be a general purpose processor, an FPGA, and/or an ASIC.

System 500 is configurable to translate analog to digital signals, and additionally to translate input video signals from a CCD to signals compatible with a control unit 550 that expects a CMOS image sensor. To accomplish this conversion, additional clock signals are required in addition to circuitry to digitize the CCD output. As can be seen, system 500 includes various clock signal generators such as vertical transfer clock generator 543, horizontal transfer clock generator 544, and substrate clock generator 545.

In the example depicted, signal translator 530 receives a CCD signal from CCD image sensor 522. A/D converter 536 is used to digitize the analog output of the CCD sensor. D/A converter 532 is used, in conjunction with adjustable voltage reference 534, to set the voltage reference of the A/D converter 536. Adjustable voltage reference 534 can be set based on the particular type of CCD sensor and/or expected CMOS output, or can be changed dynamically to adjust the image based on information within the image.

In some aspects, control unit 550 can provide a clock signal to signal translator 530. In some cases, control unit 550 can expect a pixel array of a specific size. As such, signal translator 530 can calculate clock signals to provide to CCD image sensor 522 such that the intended pixel array size is obtained.

A typical CMOS image sensor uses one input clock to drive the circuitry, whereas a CCD sensor has multiple clocks. Processor 540 can therefore receive as input a standard CMOS sensor clock that is typically generated by control unit 550, and convert this clock timing into the multiple clocks needed to drive the CCD sensor 522. In the case of an analog CCD sensor, these clocks can also provide timing for the A/D converter 536 such that the sample rate is a minimum of 1:1 with the pixel data rate. D/A converter 538 provides a reference for the A/D converter 536. This allows the FPGA to dynamically adjust the A/D reference thus providing pseudo gain and/or black level adjustment. Additional analog gain circuits can be added before A/D converter 536 if necessary. The sync generator and pixel formatter 547 aligns the data in a pattern that is expected by the control unit 550.

Typical CMOS image sensors send the data as row by row with alternating color pixels, for example row 1 would be BGBGBG repeated and row 2 would be GRGRGR repeated and so forth. The analog readout of a sensor can be quite different where all of the blue pixels in a row are sent and then all of the green pixels in the same row are sent. This would be the function of the pixel formatter to take that data and rearrange it in the row by row color pattern that would be typical of a CMOS sensor. Data formatter 547 operates to serialize the digitized and formatted data and insert synchronization. The synchronizations can be in the form of bits within a word of the pixel data. For example, if the pixel data was eight bits, the word could be ten bits with the first two bits signaling valid frame and line respectively. The data formatter 547 could also insert a start or an end of a line code or a frame code on the data along with generated data that is sent during blanking periods. This would depend on what the control unit expects for image data. The data I/O interface 548 is a circuit that connects to the control unit using a signal level expected by the control unit. The data I/O interface 548 could be single ended parallel signaling and standard CMOS voltage levels, or differential LVDS, MIPI, and so forth.

Additionally or alternatively, signal translator 530 can permit gain and/or black level adjustments.

In some cases, the pixel readout from the CCD image sensor 522 might not follow the same pattern as the expected CMOS signal at control unit 550. If so, processor 540 and/or other circuitry may be used to buffer the image data coming from the CCD image sensor 522 into a memory to allow for processing to take place.

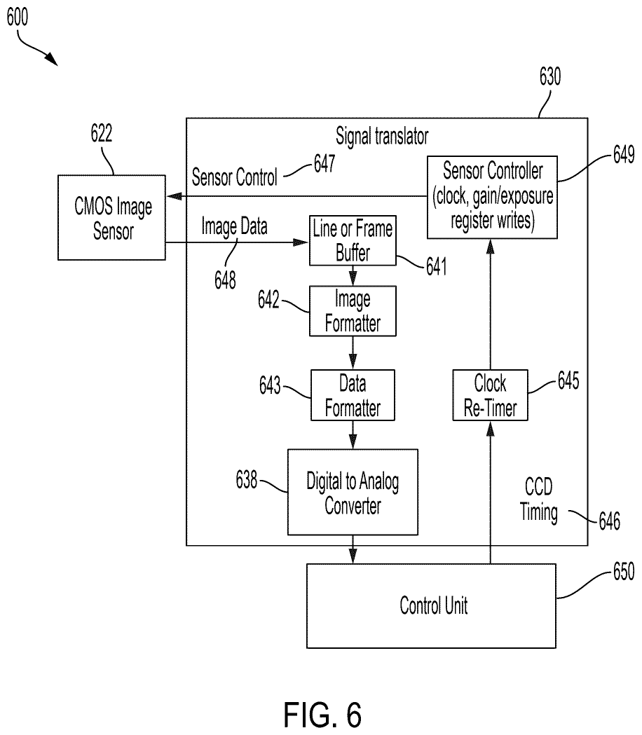

While a CMOS relies on limited clocks and has a straightforward pixel data output, a CCD output and clocking can be more complex. Furthermore, most CMOS image sensors produce a digital output while CCD sensors produce an analog signal. Accordingly, disclosed techniques include converting CMOS signals to CCD signals, for example as depicted in FIG. 6.

FIG. 6 is a diagram of a CMOS to CCD conversion system 600 for an endoscope, according to aspects of this disclosure. System 600 includes CMOS image sensor 622, signal translator 630, and control unit 650. In the example depicted, signal translator 630 translates a CMOS image signal received from CMOS sensor 622 into a CCD signal.

Signal translator 630 includes D/A converter 638, line or frame buffer 641, image formatter 642, data formatter 643, sensor controller 649, and clock re-timer 645. Signal translator 630 receives CCD timing 646 from control unit 650. CCD timing 646 is in turn passed to clock re-timer 645, which passes a signal to sensor controller 649.

CMOS image sensor 622, when provided control signal 647, outputs image data 648. Image data 648 is passed to line or frame buffer 641, which buffers a predetermined amount of data (e.g., a line or a frame), and passes that predetermined amount of data to image formatter 642. Image formatter 642 in turn passes a formatted image to data formatter 643, which passes data to D/A converter 638. In turn, the analog data from D/A converter 638 is passed to control unit 650.

In some cases, sensor controller 649 in turn generates sensor control signal 647 to CMOS image sensor 622. For instance, in some aspects, signal translator 630 can determine a physical size of the array of CMOS image sensor 622 and write one or more appropriate register commands to the CMOS image sensor 622 to specify a size of the image array. In some aspects, storage may be required to buffer a line or frame of image and timing data is recreated as expected by the control unit 650. For instance, the signal translator 630 can read one or more clocks from control unit 650 such that appropriate commands can be sent to CMOS image sensor 622 such that CMOS image sensor 622 is configured to generate an image with the appropriate dimensions.

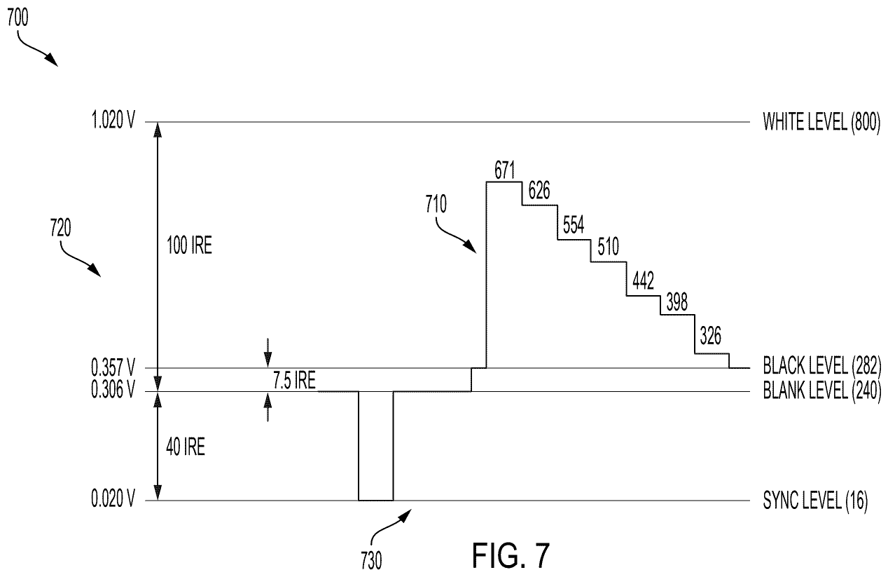

Different analog sensors may also rely on different signaling schemes. In some cases, therefore, certain aspects involve converting between different analog signals, an example of which is shown in FIG. 7.

FIG. 7 is a diagram of example analog signaling schemes for use in an endoscope, according to aspects of this disclosure. FIG. 7 depicts plot 700, which includes an analog signal 710 plotted with respect to voltage 720 and time 730.

Plot 700 represents a digitized National Television Standards Committee (NTSC) signal. Analog video can have a base level to which syncs and data are referenced. The base level is usually positive with respect to the sync pulse and is usually close to the level that represents black in the active video data. Signal 710 can represent an entire line of video with timing, brightness, and color information.

In some cases, video translators described herein can digitize analog signals, for example, generated from a non-NTSC based analog sensor and then convert the digital signal to an NTSC-compatible analog signal. In some cases, data received from an image sensor may include pixel values corresponding to gray levels, rather than colors. In these cases, demosaicing, or color reconstruction, can be performed, as discussed herein.

Referring back to FIGS. 3 and 4, system 300 can be used to convert analog to digital video and system 400 to convert digital to analog video. Systems 300 and 400 can also convert between analog formats. For example, a signal translator of system 300 or 400 can employ an A/D converter to digitize an input NTSC signal. The signal translator then processes the digitized signal and translates the signal back to a different analog signal. Examples of processing include cropping and resizing of the digitized image.

For instance, system 300 or 400 can use sync detection circuitry that can be used to detect a negative or positive going sync pulse. The adjustable reference allows detection of sync pulses of many different voltage levels. Similar voltage reference circuitry is also used for the analog to digital converter that will allow digitization of many different analog video voltage levels.

For control units that expect particular analog video input, an analog image sensor that has a different format can be digitized and the data passed into an image processor. Then, the data can be converted back to analog video, with care paid to timing and sync levels expected by the control unit.

Different digital image sensors using different video signaling schemes may also be converted. For example, LVDS and MIPI signaling, while both use differential signaling, are incompatible. MIPI refers to a signal that consists of a high-speed (HS) portion and a low power (LP) portion. The HS portion is a differential signal with a common mode voltage typically around 200 millivolts (mV) and a differential mode voltage around 200 mV (each line swings +/−100 mV). The LP mode voltage is driven as single ended on the differential lines at a voltage of about 1.2 V. LVDS refers to a differential signal with a common mode voltage of approximately 1.2 V and a differential voltage of approximately 350 mV. Conversion to LVDS allows data to be transmitted a long distance (3 meters or more) to a receiver.

Accordingly, disclosed systems can convert from an LVDS signaling scheme to a MIPI signaling scheme and vice versa.

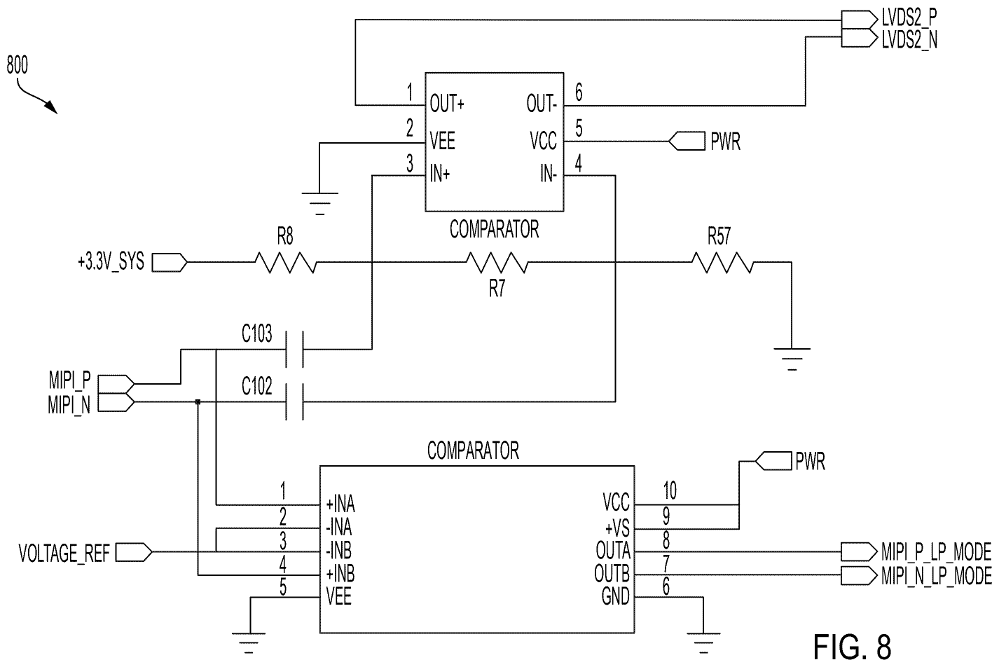

FIG. 8 is a diagram of an example circuit 800 for a Mobile Industry Processor Interface (MIPI) to Low Voltage Differential Signaling (LVDS) conversion system for an endoscope, according to aspects of this disclosure. Circuit 800 is configurable to convert an LVDS signal from an LVDS-compatible image sensor (not shown) to a signal expected by a MIPI-compatible control unit (not shown). Circuit 800 can include signal level shifting and/or amplification. Additionally, timing may be adjusted between the two different sides of the translation.

Circuit 800 converts high-speed MIPI differential mode data to high-speed LVDS data, and converts the low power single-ended MIPI control signals to control signals for an image processor to use. Circuit 800 in combination with a processor such as an FPGA or ASIC can convert a single or multi-lane MIPI sensor to a single or multi-lane LVDS format for a control unit that expects LVDS.

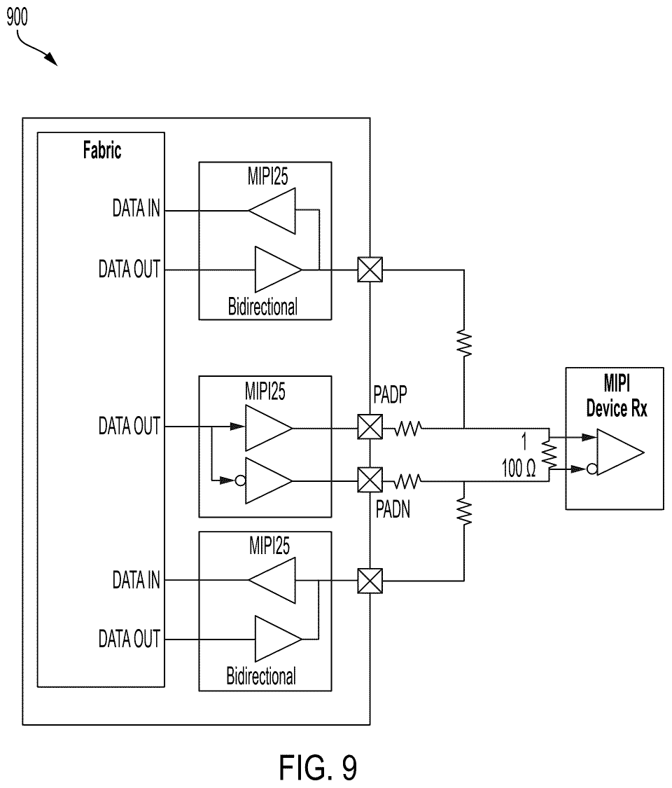

Conversely, a control unit that expects a MIPI type sensor can use an LVDS sensor with a circuit as depicted in FIG. 9.

Circuit 800 includes a high-speed comparator that outputs a LVDS signal. The comparator can have a single ended output or a differential output. Circuit 800 also includes two lower-speed comparators, each with a single ended output. The lower speed comparators could have a differential output. During the HS portion of the MIPI signal, the high-speed comparator repeats the incoming signal and converts the signal level to LVDS.

During the HS portion, the lower speed comparator's outputs are zero since the HS signaling is well below a reference voltage. The reference voltage can safely be set to a voltage between 400 mV to 800 mV depending on the application. When the MIPI signal enters the LP mode, the 1.2 V signal swing will trigger the lower speed comparator thereby giving a single ended output for each of the differential lines. Each output can be converted to differential if the signal is required to travel a long distance or can be kept as is. The LP signal typically includes frame and line start and stop information used for synchronization. The HS portion contains the image data. Circuit 800 can be constructed with or without an FPGA or an ASIC. In some cases, a microprocessor or GPU could also be used as the receiver after circuit 800.

FIG. 9 is a diagram of an example circuit 900 for a LVDS to MIPI conversion system for an endoscope, according to aspects of this disclosure. Circuit 900 receives an output from a LVDS image sensor (not shown). The image data is driven out to a differential driver for the MIPI high-speed signaling. As can be seen, bidirectional drivers are connected to each the N and P channels of the MIPI differential drive. When the horizontal or vertical syncs are received from the LVDS sensor, the FPGA or ASIC places the high-speed differential driver into a high impedance state and drives each of the N and P channels independently for the correct control of the MIPI LP mode. FIG. 9 depicts a 100 Ohm resistor, which can be an on or off chip.

Additionally or alternatively, different type of digital image sensor data conversion may be required, for example, to convert from a digital LVDS signal with an embedded clock signal, to a digital LVDS signal without an embedded clock signal. The LVDS signal can, in this case, for example be routed into an FPGA. The FPGA can decode the clock signal from the LVDS data, and send the data, without the embedded clock to the control unit. Care needs to be taken that the sync signals required by the existing control unit can be matched.

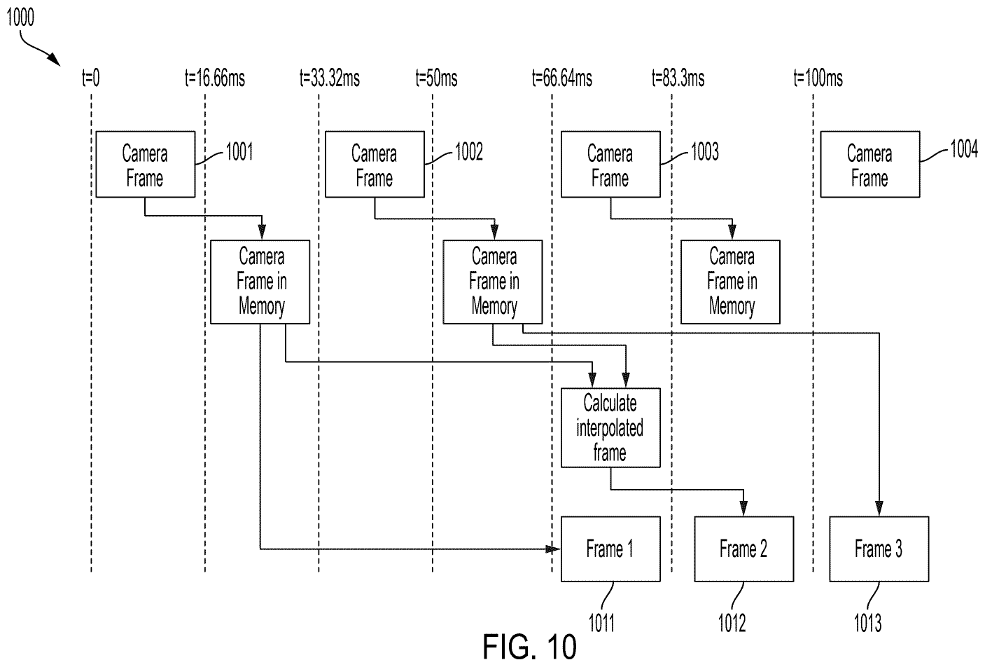

In some aspects, frame rate conversion is performed before a signal is passed to the control unit. For instance, a video sequence with a frame rate of 60frames/sec (fps) may need to be converted to 30 fps or vice versa. A frame rate down conversion could be executed by dropping a complete frame as needed to achieve the desired lower frame rate. In the given example from 60 fps down to 30 fps, every other frame would be dropped, however, if the desired frame rate was different, a different number of frames could be dropped. For example, for a conversion from 60 fps to 45 fps, 1 out of 4 frames could be dropped. FIG. 10 depicts an example of frame rate conversion.

Circuit 900 could be built with discrete components or with an FPGA or an ASIC. In an example, the FPGA would receive incoming video data. In this manner, the pixel data along with the horizontal and vertical syncs are known. The output of the FPGA is a tri-stateable differential driver along with two tri-stateable single ended output drivers. The FPGA can control tri-stateable outputs for driving the HS and LP modes. The FPGA can have a timing generator that would send both modes in the speed and timing that the receiver would expect. For example, if the receiver expected data at a rate that would be associated with a large sensor, but the sensor connected to the FPGA is a small sensor, the FPGA can insert artificially created data to fill the timing void in the data channel, but still provide LP mode information at the timing expected of a large sensor.

FIG. 10 is a flow diagram 1000 of an example frame rate conversion system for an endoscope, according to aspects of this disclosure. Flow diagram 1000 depicts multiple camera frames, each of which is received at 33.32 millisecond intervals, making a frame rate of 30 fps. Flow diagram 1000 represents a conversion to double the frame rate, specifically 60 fps.

In an example, to perform frame rate conversion, a processor receives a first video signal having a first frame rate and converts the first video signal to a second video signal having a second frame rate. As depicted in flow diagram 1000, at time 0 milliseconds (ms), camera frame 1001 is received and stored in memory. At time 33.32 ms, camera frame 1002 is received and is stored in memory. At time 66.64 ms, camera frame 1003 is received and stored in memory. Additionally, camera frames 1001 and 1002 are retrieved from memory and camera frame 1011 is output, reflecting a processing delay of 66.64 ms, and an interpolated frame that represents a frame between frame 1001 and 1002 is calculated. At time 83.3 ms, the interprolated frame is output, and so forth. In some cases, a simple duplication of frames is used for interpolated frames, which simplifies processing.

Interpolation of video frames involves creating a new frame from existing frames such that the new frame is spatially and temporally coherent with the existing frames. Different algorithms are possible, including adaptive and fixed approaches.

Additionally or alternatively, certain aspects can perform de-mosaicing, or color reconstruction, of image signals. Pixel data obtained from an image sensor may have an incomplete set of pixel data for an image. For example, such pixel data may have a subset of the total pixel data for each of red, green, and blue measurements. By contrast, de-mosaiced image data includes a pixel value corresponding to each pixel in an array for each of red, green, and blue. Disclosed techniques can perform this conversion, for example using an FPGA, ASIC, or other image processing device.

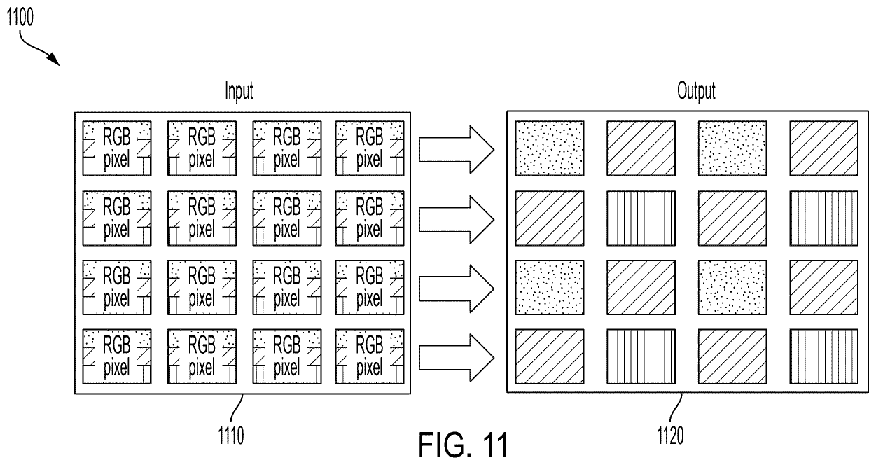

Further, disclosed techniques can perform remosaicing of data, for example, if a control unit expects mosaiced data but the image sensor provides RGB de-mosaiced data, then the signal translator can re-mosaic the signal. In an example, to re-mosaic, the representative R, G, or B component of the RGB pixel data is extracted and used for frame data. Other approaches are possible. FIG. 11 depicts inputs and outputs for a re-mosiacing process.

FIG. 11 is a diagram 1100 of inputs and outputs of a video re-mosaicing system for an endoscope, according to aspects of this disclosure. Diagram 1100 depicts raw data 1110 and mosaiced image data 1120. As can be seen, data 1110 includes each pixel having a value for each of R, G, and B data. By contrast, mosaiced image data 1120 includes an output sequence that includes R, G, and B as separate data values in an interweaved fashion. Different types of re-mosaicing algorithms may be used.

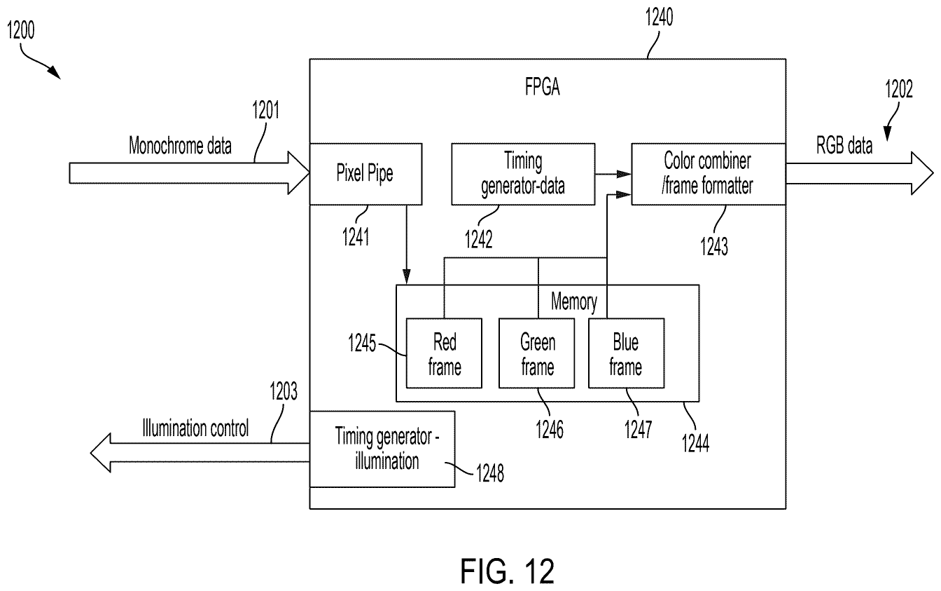

Certain aspects can translate between monochrome and RGB or vice versa. FIG. 12 depicts an example of one such system.

FIG. 12 is a diagram of an example monochrome to color conversion system 1200 for an endoscope, according to aspects of this disclosure. System 1200 includes FPGA 1240. In the example depicted, FPGA 1240 generates RGB data 1202 by activating a light source via illumination control 1203 and receiving monochrome data 1201 captured while the image sensor is appropriately illuminated. While the RGB color space is used for illustrative purposes, conversion to or from any color space is possible.

FPGA 1240 includes pixel pipe 1241, timing generator-data 1242, color/frame combiner 1243, memory 1244 including red frame 1245, green frame 1246, and blue frame 1247, and timing generator-illumination 1248. FPGA 1240 operates by generating repeating groups of three frames, where each frame is generated using red, green, and blue illumination respectively. Data from the three frames is combined to create the desired RGB data.

Memory 1244 is used to store intermediate data. Memory 1244 may or may not be within the FPGA 1240. FPGA 1240 can generate the timing necessary to synchronize the differently colored illumination with the exposure of the camera frames. Pixel pipe 1241 reads the incoming data and stores the incoming data in memory 1244. Each incoming data point is assigned to the corresponding frame and correct pixel location. Color/frame combiner 1243 receives data from all three frames to generate one frame that has RGB data per pixel. Timing generator-illumination 1248 is operable to pulse the correct light (e.g., red, green, or blue). The timing of the pulses is coordinated to match the expected image within such that the red, green, and blue frame are assigned correctly.

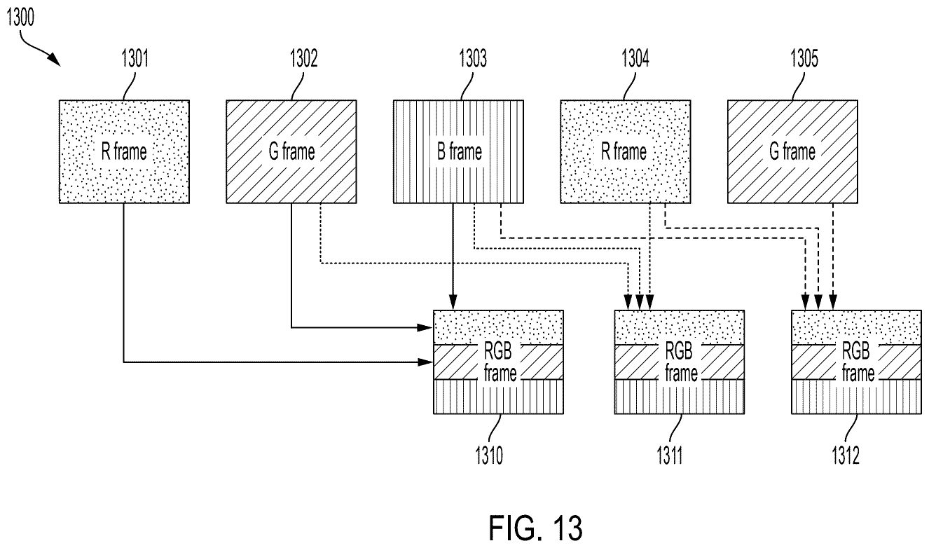

FIG. 13 is a diagram 1300 of exemplary inputs and outputs of a monochrome to color conversion system for an endoscope, according to aspects of this disclosure. RGB frames are formed from individual R, G, and B frames.

As can be seen, R frame 1301, G frame 1302, and B frame 1303 are combined into RGB frame 1310. G frame 1302, B frame 1303, and R frame 1304 are combined into RGB frame 1311. B frame 1303, R frame 1304, and G frame 1305 are combined into RGB frame 1312.

Diagram 1300 depicts a rolling-frame approach. For instance, each sequential RGB output frame is created from a rolling (or sliding) window of a sequential triplet of R, G, and B input frames. A number of RGB frames equal to the total number of R, G, and B frames combined is created. In some cases, a processor can be used to smooth out motion artifacts created by the conversion process.

Various aspects relate to adaptors that facilitate interoperability between endoscopes and control units. For an adapter (including external equipment not integrated into the endoscope) to be useful for use with existing control units and a new or different endoscope not formerly used with that control unit, it needs to be safely integrated into existing equipment. Typically, in endoscopes that use optical/illumination fibers to transmit light to the distal tip, an endoscope is securely connected to the light source via an umbilicus to couple light from the light source into the illumination fibers, and an electrical connection (e.g., a pigtail) is used to connect the electrical circuitry to the image processor.

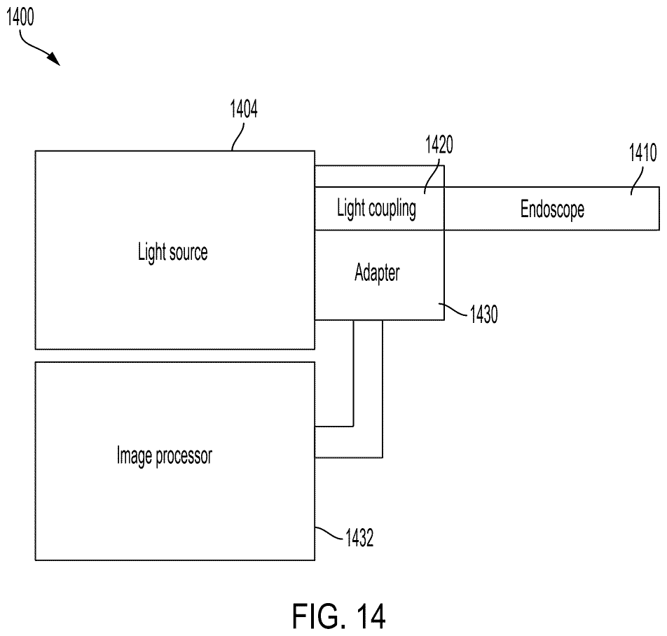

The video converters described herein may be attached to a light source that is not positioned at the end of the endoscope but rather in the control unit. In that case, an adaptor may be used that contains an illumination fiber assembly, allowing for light to be coupled from the existing control unit's light source through the adapter, into the endoscope. FIG. 14 depicts one such example.

FIG. 14 is a diagram of an example mechanical adaptor system 1400 for use with an endoscope and a signal translator, according to aspects of this disclosure. System 1400 includes endoscope 1410, light source 1404, light coupling 1420, adaptor 1430, and image processor 1432. As can be seen, light coupling 1420 connects light source 1404 to endoscope 1410 such that light passes from light source 1404, through the light coupling 1420 of adapter 1430, to endoscope 1410. Adaptor 1430 can house one or more components of a video signal translator, and, as shown, is between endoscope 1410 and image processor 1432.

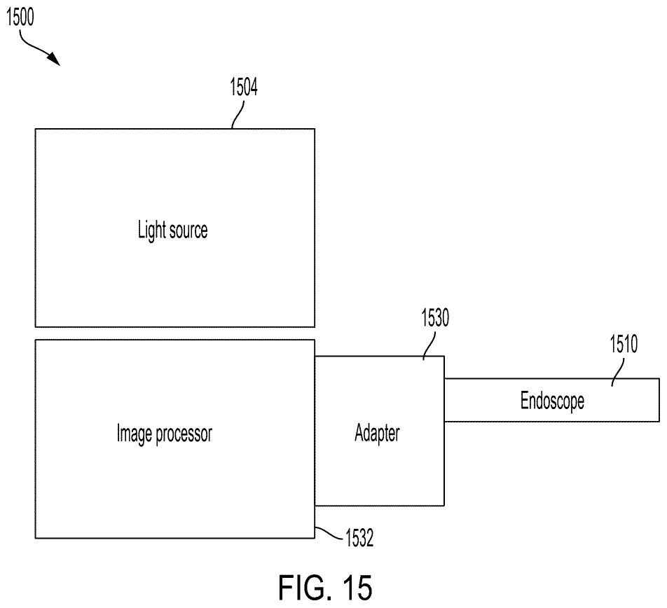

FIG. 15 is a diagram of an example mechanical adaptor system 1500 for use with an endoscope and a signal translator, according to aspects of this disclosure. System 1500 includes endoscope 1510, light source 1504, adaptor 1530, and image processor 1532. In system 1500, adaptor 1530 is attached to a pig tail port on the image processor 1532 as the light source 1504 is not required for the system to function as intended. For example, endoscope 1510 may include one or more LED(s) at a distal end as a light source, and an umbilicus connects endoscope 1510 to adapter 1530. Adapter 1530 in turn connects to a port of image processor 1532, which may provide electrical signals for the LEDs along with image processing functionality.

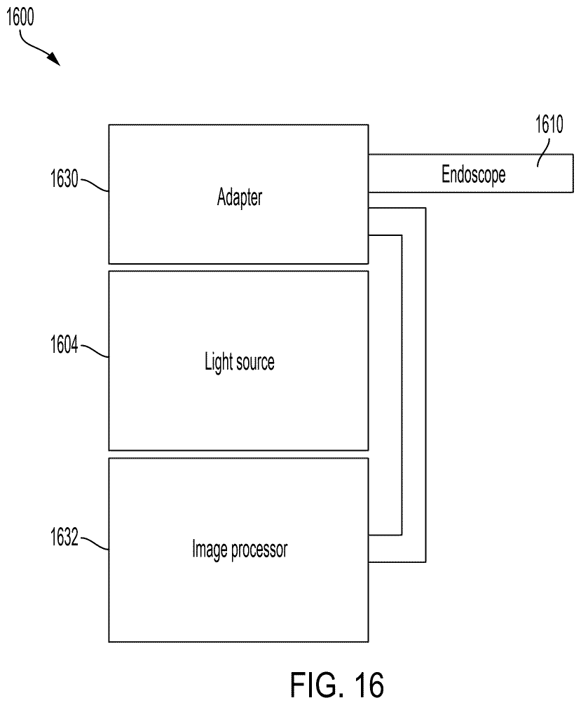

FIG. 16 is a diagram of an example mechanical adaptor system 1600 for use with an endoscope and a signal translator, according to aspects of this disclosure. System 1600 includes endoscope 1610, light source 1604, adaptor 1630, and image processor 1632. In system 1600, the converter (adaptor 1630) is a stand-alone piece of equipment that is added to the equipment stack of the image processor 1632 and light source 1604. Like system 1500, endoscope 1610 may include one or more LED(s) at a distal end as a light source, and an umbilicus connects endoscope 1610 to adapter 1630. Adapter 1630 in turn connects to a port of image processor 1632, for example via an umbilicus (or other electrical cabling) of adapter 1630. Image processor 1632 may provide electrical signals for the LEDs along with image processing functionality.

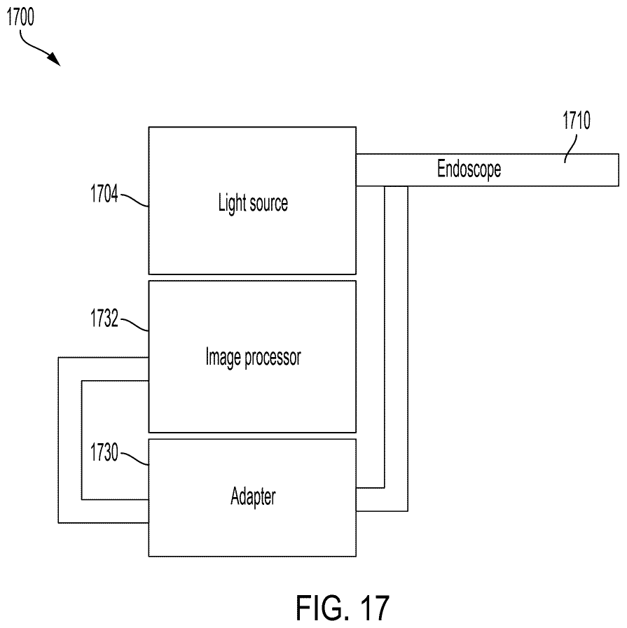

FIG. 17 is a diagram of an example mechanical adaptor system 1700 for use with an endoscope and a signal translator, according to aspects of this disclosure. System 1700 includes endoscope 1710, light source 1704, adaptor 1730, and image processor 1732. In system 1700, adapter 1730 is a piece of equipment added to the stack, however, the existing equipment's light source 1704 is still being used to provide illumination to endoscope 1710, for example via illumination fiber technology.

In another aspect, the image sensor translator resides within the endoscope. This may be in the connector used to connect the endoscope to the existing piece of equipment, or within the handle of the endoscope.

FIG. 18 depicts an example of a computing device 1800, according to aspects of this disclosure. FIG. 18 is a simplified functional block diagram of computing device 1800 that may be configured as a device for executing processes described herein. In various aspects, any of the systems herein may be or include computing device 1800 including, e.g., a data communication interface 1820 for packet data communication. Computing device 1800 may communicate with one or more other computers, for example, using an electronic network 1825 (e.g., via data communication interface 1820). Electronic network 1825 may include a wired or wireless network.

Computing device 1800 also may include a central processing unit (“CPU”), in the form of one or more processors 1802, for executing program instructions 1824. Program instructions 1824 may include at instructions for functions such as controlling components of various systems described herein such as D/A or A/D controllers and/or performing image processing

Computing device 1800 may include an internal communication bus 1808. Computing device 1800 may also include a drive unit 1806 (such as read-only memory (ROM), hard disk drive (HDD), solid-state disk drive (SDD), etc.) that may store data on a computer readable medium 1822 (e.g., a non-transitory computer readable medium), although computing device 1800 may receive programming and data via network communications. Computing device 1800 may also have a memory 1804 (such as random-access memory (RAM)) storing instructions 1824 for executing techniques presented herein. It is noted, however, that in some aspects, instructions 1824 may be stored temporarily or permanently within other modules of computing device 1800 (e.g., processor 1802 and/or computer readable medium 1822). Computing device 1800 also may include user input and output devices 1812 and/or a display 1810 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical system comprising:
a video signal translator configured to:
receive a first video signal from an imaging device, the first video signal having a first video signal format;
translate the first video signal from the first video signal format to a second video signal format that is different from the first video signal format; and
output, to a control unit, the translated video signal as a second video signal, wherein the control unit is configured to receive the second video signal and process the second video signal for output to an electronic display.

2. The medical system of claim 1, further comprising a medical device including a distal tip portion, the distal tip portion including the imaging device configured to output the first video signal in the first video signal format.

3. The medical system of claim 2, wherein the video signal translator is positioned within a handle of the medical device.

4. The medical system of claim 1, wherein:
the first video signal format is an analog video format,
the second video signal format is a digital video format,
the video signal translator comprises an analog-to-digital converter, and
translating the first video signal comprises:
applying the analog-to-digital converter to the first video signal, and
receiving, from the analog-to-digital converter, the second video signal.

5. The medical system of claim 4, wherein the imaging device is a Charge Coupled Device (CCD), and wherein the second video signal is compatible with a signal from a metal oxide semiconductor (CMOS).

6. The medical system of claim 1, wherein:
the first video signal comprises a first frame rate, and
the system further comprises a processor that is configured to adjust the first frame rate of the first video signal to a second frame rate of the second video signal, wherein the second frame rate is different than the first frame rate.

7. The medical system of claim 2, further comprising a lighting source configured to cause light to be emitted at the distal tip portion, and wherein the translation of the first video signal to the second video signal comprises:
causing the lighting source to emit, at a first time, light having a first primary color;
capturing a first frame of video;
causing the lighting source to emit, at a second time, light having a second primary color different from the first primary color;
capturing a second frame of video; and
constructing the second video signal from the first frame of video and the second frame of video.

8. The medical system of claim 1, wherein the system further comprises a processor that is configured to deconstruct the first video signal into separate primary color components and construct the second video signal with the primary color components into a mosaic, wherein each pixel value of the second video signal comprises a single primary color component.

9. The medical system of claim 1, wherein
the first video signal format is a digital signal format,
the second video signal is an analog signal format;
the video signal translator comprises a digital-to-analog converter, and
translating the first video signal comprises:
applying the digital-to-analog converter to the first video signal, and
receiving, from the digital-to-analog converter, the second video signal.

10. The medical system of claim 9, wherein the imaging device is a CMOS device, and wherein the second video signal is compatible with a signal from a CCD.

11. The medical system of claim 1, wherein the first video signal format is an analog video signal format with a first signaling type, and the second video signal format is a second analog video signal format having a second signaling type that is different the first signaling type.

12. The medical system of claim 1, wherein each of the first video signal format and the second video signal format conform to a Low Voltage Differential Signaling (LVDS) format or a Mobile Industry Processor Interface (MIPI) format.

13. The medical system of claim 2, further comprising an adaptor having the video signal translator, wherein the adaptor is attachable to the medical device.

14. The medical system of claim 13, wherein the medical device is an endoscope, and wherein the adaptor comprises a light coupling configured to propagate light from a lighting source to the endoscope.

15. The medical system of claim 1, wherein the control unit is configured to process the second video signal prior to outputting the second video signal to an electronic display.

16. A medical system comprising:
a medical device including a shaft having a distal tip portion, the distal tip portion including an imaging device configured to output a first video signal in a first video signal format; and
a video signal translator configured to:
receive the first video signal from the imaging device;
translate the first video signal from the first video signal format to a second video signal format that is different from the first video signal format; and
a control unit configured to:
receive the translated video signal from the video signal translator, and
process the translated video signal into an image processed video signal for output.

17. The medical system of claim 16, wherein:
the first video signal format is an analog video format,
the second video signal format is a digital video format,
the video signal translator comprises an analog-to-digital converter, and
translating the first video signal comprises:
applying the analog-to-digital converter to the first video signal, and
receiving, from the analog-to-digital converter, the second video signal.

18. The medical device system of claim 16, wherein
the first video signal format is a digital signal format,
the second video signal is an analog signal format;
the video signal translator comprises a digital-to-analog converter, and
translating the first video signal comprises:
applying the digital-to-analog converter to the first video signal, and
receiving, from the digital-to-analog converter, the second video signal.

19. A method of operating a medical device, the method comprising:
receiving, from an imaging device within the medical device, a first video signal in a first video signal format;
translating the first video signal from the first video signal format to a second video signal format that is different from the first video signal format;
outputting, to a control unit, the translated video signal as a second video signal;
receiving, at the control unit, the second video signal;
processing the second video signal to an image processed video signal, via the control unit; and
displaying, on an electronic display, the image processed video signal.

20. The method of claim 19, wherein:
the first video signal format is an analog video format,
the second video signal format is a digital video format, and
translating the first video signal comprises:
applying an analog-to-digital converter to the first video signal, and
receiving, from the analog-to-digital converter, the second video signal.
